(19) United States
(12) Patent Application Publication
(10) Pub. No.: US 2016/0339063 A1
(43) Pub. Date: Nov. 24, 2016

(54) COMPOSITION FOR PREVENTING AND HEALING COMPROMISED BONE AND A METHOD OF MAKING SAME

(71) Applicants: Dmitriy G. Elistratov, Penza (RU); Vilorij I. Strukov, Penza (RU); Mikhail D. Prokhorov, Penza (RU); Olga Jones-Strukova, Grand Praire, TX (US); Vyacheslav N. Trifonov, Zarechny (RU); Julija A. Elistratova, Penza (RU); Konstantin G. Elistratov, Penza (RU); Natal'ja V. Kurus', Penza (RU); Natalya V. Eremina, Penza (RU); Marina M. Maksimova, Penza (RU); Ramziya T. Galeeva, Penza (RU); Larisa G. Radchenko, Penza (RU); Aleksandr V. Fedorov, Kuznetsk (RU); Evgeny N. Krutyakov, Penza (RU); Elena S. Andreeva, Penza (RU); Tatiana V. Elistratova, Penza (RU); Irina V. Khomyakova, Penza (RU); Galina A. Tolbina, Penza (RU)

(72) Inventors: Dmitriy G. Elistratov, Penza (RU); Vilorij I. Strukov, Penza (RU); Mikhail D. Prokhorov, Penza (RU); Olga Jones-Strukova, Grand Praire, TX (US); Vyacheslav N. Trifonov, Zarechny (RU); Julija A. Elistratova, Penza (RU); Konstantin G. Elistratov, Penza (RU); Natal'ja V. Kurus', Penza (RU); Natalya V. Eremina, Penza (RU); Marina M. Maksimova, Penza (RU); Ramziya T. Galeeva, Penza (RU); Larisa G. Radchenko, Penza (RU); Aleksandr V. Fedorov, Kuznetsk (RU); Evgeny N. Krutyakov, Penza (RU); Elena S. Andreeva, Penza (RU); Tatiana V. Elistratova, Penza (RU); Irina V. Khomyakova, Penza (RU); Galina A. Tolbina, Penza (RU)

(21) Appl. No.: 15/230,746

(22) Filed: Aug. 8, 2016

Related U.S. Application Data
(63) Continuation-in-part of application No. 13/512,671, filed on May 30, 2012.
Application No. 13/512,671 is a continuation-in-part of application No. 14/760,699, filed on Jul. 13, 2015.
Application No. 14/760,699 is a continuation-in-part of application No. 14/760,702, filed on Jul. 13, 2015.

(30) Foreign Application Priority Data
Nov. 30, 2009 (RU) 2009144461
Jan. 11, 2013 (RU) 2013100994
Jan. 11, 2013 (RU) 2013100995

(51) Int. Cl. A61K 35/64; A61K 33/10; A61K 33/06

(52) U.S. Cl. CPC A61K 35/64; A61K 33/06; A61K 33/10

(57) ABSTRACT

A biologically active additive composition for preventing and healing bone diseases comprises a pharmacologically active compound of calcium chosen from a series comprising calcium carbonate, calcium citrate, calcium gluconate, calcium aspartate, calcium amino acid chelate, calcium fumarate, calcium succinate, calcium ascorbate, calcium phosphate, or any combination thereof, in the amount of 16.67-93.75 weight %, and drone brood in the amount of 6.25-83.33 weight %. Due to the combination, osteoporosis, bone fractures and cavities, arthritis, arthrosis, paradontosis can be prevented and healed with substantially reduced risk of calcium deposition in soft tissues. Drone brood can be in the form of an adsorbed or lyophilised homogenate. Methods of making the composition are also disclosed.

### CROSS-REFERENCE TO RELATED APPLICATIONS

This application is a continuation in part of: (1) U.S. application Ser. No. 13/512,671 filed on May 30, 2012, pending, which is a U.S. National Stage application of International application PCT/RU2010/000613 filed on Oct. 21, 2010, which claims priority of Russian application RU 2009144461 filed on Nov. 30, 2009; (2) U.S. application Ser. No. 14/760,699 filed on Jul. 13, 2015, pending, which is a U.S. National Stage application of International application PCT/RU2010/000008 filed on Jan. 13, 2014, which claims priority of Russian application RU 2013100995 filed on Jan. 11, 2013; and (3) U.S. application Ser. No. 14/760,702 filed on Jul. 13, 2015, pending, which is a U.S. National Stage application of International application PCT/RU2010/000009 filed on Jan. 13, 2014, which claims priority of Russian application RU 2013100994 filed on Jan. 11, 2013. All nine above applications are incorporated herein by reference in their entirety.

### BACKGROUND OF THE INVENTION

1. Field of the Invention

A composition is proposed which prevents developing, and assists in fighting, progressing arthritis, arthrosis, paradontosis, accelerates fractured bone union, substantially lowers fracture risk, facilitates regeneration of the bone tissue in osteoporosis without risk of calcium sedimentation in soft tissues, and promotes filling cavities in bone tissues.

2. Description of Related Art

Closest analogs to the proposed composition are:
“Apilar” tablets based on drone brood (patent RU2233666, publ. Aug. 10, 2004)—a food additive comprising 12-14.5% of drone brood homogenate and also comprising calcium stearate;
plant extracts intended for treating bone resorption (patent RU2196601, publ. Jan. 20, 2003)—a food additive for preventing osteoporosis and comprising physiologically acceptable calcium source at a rate of 5-50 mass %, as well as other active components such as vitamin D and plant estrogens;
calcium-related information (deficit, use in additives, benefits, side effects—http://www.zhion.com/Supplements/Calcium.html, posted Aug. 23, 2013).

The use of all the compositions identified above for preventing osteoporosis and assisting in other bone tissue diseases fails to avert calcium sedimentation in soft tissues—calcification.

### SUMMARY OF THE INVENTION

The object of the present invention is to provide a composition able to prevent and heal such compromised bone manifestations as osteoporosis, bone fractures and cavities, arthritis, arthrosis, paradontosis, with substantially reduced risk of calcium deposition in soft tissues.

The main technical result of the composition is fixating calcium in the body, when administered, with lowering the risk of calcium deposition in untargeted organs.

The above object with the above technical result are achieved by providing a composition (a biologically active additive, BAA) comprising a calcium compound chosen from a series comprising calcium carbonate, calcium citrate, calcium gluconate, calcium aspartate, calcium amino acid chelate, calcium fumarate, calcium succinate, calcium ascorbate, calcium phosphate, or any combination thereof, in the amount of 16.67-93.75 weight % and drone brood in the amount of 6.25-83.33 weight %.

To compound the BAA, an adsorbed drone brood homogenate or a lyophilisated drone brood is used. The adsorbed drone brood is made by extracting drone brood from a honeycomb and milling/grinding same; providing one part of drone brood homogenate and 3-30 parts of an adsorbent by mass, wherein said adsorbent is selected from the group comprising lactose, glucose, fructose, or any combination thereof; mixing the one part of drone brood homogenate with the 3-30 parts of adsorbent: vacuum drying the mixture of the homogenate and adsorbent, which starts no later than 55 minutes after extracting drone brood from the honeycomb, progresses without heating the mixture and with pressure in the vacuum device no less than 1 mm Hg, and ends at the humidity of the product not exceeding 1.5%. To prepare the lyophilisate, drone brood larvae are homogenized, for example in a mixer, and are freezed for 2-3 hours at −35÷−40° C. Then, the product is subjected to vacuum sublimation at a residual pressure of 0.1÷0.6 mm Hg during 40-48 hours, with the temperature reaching 25÷30° C. by the end of the lyophilisation. The calcium compound is then milled and mixed with the drone brood to homogeneity, and tablets or capsules are prepared therefrom.

The invention is further discussed in more detail.

### DETAILED DESCRIPTION OF THE INVENTION

The claimed BAA is based on an adsorbed drone brood homogenate or a lyophilisated drone brood. Adsorption of the homogenate preserves tertiary and quaternary structure of proteins and thus drone brood hormones are better preserved. The adsorbed drone brood is prepared by mixing the homogenate with carbohydrates serving as preservatives. The adsorbed drone brood is made by extracting drone brood from a honeycomb and milling/grinding same into a homogenate. Then, it is provided one part of the obtained drone brood homogenate and 3-30 parts of a carbohydrate adsorbent by mass. The adsorbent is selected from the group comprising lactose, glucose, fructose, or any combination thereof. The one part of drone brood homogenate is mixed with the 3-30 parts of adsorbent, and the mixture is subjected to vacuum drying. This stage must start no later than 55 minutes after extracting drone brood from the honeycomb, after which time the quality of the drone brood deteriorates. The process of drying progresses without heating the mixture and with pressure in the vacuum device no less than 1 mm Hg. It ends at the humidity of the product not exceeding 1.5%. Altogether, this process is performed as disclosed in US 20150030640 A1, which application is incorporated herein in its entirety by reference. On the other hand, to prepare the lyophilisate, drone brood larvae are homogenized, for example in a mixer. The homogenate comprises a thick yellow creamy liquid with a characteristic odor. The content of dry matter in the homogenate does not exceed 22.5%. It is then freezed for 2-3 hours at −35÷−40° C. After that, the product is subjected to vacuum sublimation at a residual pressure of 0.1-0.6 mm Hg during 40-48 hours, with the temperature reaching 25÷30° C. by the end of the lyophilisation. The lyophilisate includes a powder whose color varies from beige to yellow, with a high hygroscopicity and residual moisture content of 1-3.5%. In other words, the preparation conditions of lyophilisate are such that the solid matter composition of the drone brood homogenate may be preserved without changes except for the moisture content. At the same time, the calcium compound is milled and then mixed to homogeneity with the drone brood, made by either of the above-described processes, and powder, tablets or capsules are prepared therefrom.

I. The Proposed BAA can be Used for Preventing and Healing Osteoporosis.

Known are three classes of medications for healing osteoporosis: 1) inhibitors of the absorption of bone tissue, intended for preventing bone mass from further lowering; 2) promoters of the formation of bone tissue, which include androgens and other components aimed at enhancing bone mass; and 3) promoters of bone mineralization that include a calcium absorption stimulator and calcium additives intended to increase calcium absorption and enlarge bone mass. Each of those classes has intrinsic disadvantages when healing osteoporosis.

For example, inhibitors of the absorption of bone tissue increase the risk of cancer. Banned for this reason, for example, was Miacalcic (calcitonin salmon), one of widely used preparations. Another group of inhibitors—bisphosphonates—showed low therapeutical activity when used without calcium preparations. Using androgens may cause problems associated with suppressing production of endogenic androgens. Bone tissue formation promoters have such serious side effect as calcium deposition in soft tissue, blood vessels, kidneys.

It is noteworthy that the above-mentioned bone formation promoters usually use a combination of calcium and a stimulator of calcium absorption (such as D group vitamin) which facilitates calcium absorption in the intestine and in the absence of which calcium is not absorbed (see, for example, http://nof.org/calcium#VITAMINDAREYOUATRISK—<<Vitamin D plays an important role in protecting your bones, and your body requires it to absorb calcium.>>

When administered, calcium can come not only into bones but also in other organs and systems. Specifically, it can calcify concrements in kidneys which adversely affects normal functioning thereof. The deposition of calcium in vessels results in their fragility increasing the risk of stroke and heart attack. Also, calcium deposits in other soft tissues such as muscles, brain, endocrine glands. In consuming calcium, the main factor is not so much its amount as the ability of the body to ingest calcium and use it as intended. Therefore, treating osteoporosis with preparation of calcium carries risk of having other organs calcified. Up to now, this problem was waiting for its solution. It cannot be solved by just diminishing the dosage to thus lower the risk of calcifying soft tissues because when lowering the dosage of calcium one cannot confront osteoporosis, and, consequently, the risk of fracture increases for that reason. So prior to the current proposal, a patient faced a dilemma when using calcium preparations to heal osteoporosis: to die either of osteoporosis because of hip fracture as a result of insufficient intake of calcium, or of stroke or cardiac infarction as a result of calcification of soft tissues because of excessive intake of calcium (see in this regard, for example http://heart.bmj.com/content/98/12/920.abstract—<<Increasing calcium intake from diet might not confer significant cardiovascular benefits, while calcium supplements, which might raise myocardial infarction risk, should be taken with caution.>>; http://www.medicalnewstoday.com/articles/255832.php—<<High Calcium Supplement Intake Raises Male Cardiovascular Disease Death Risk>>); http://news.bbc.co.uk/2/hi/health/7187265.stm—<<Calcium supplements may increase the risk of a heart attack in older women, New Zealand research suggests.>>, etc.) Indeed, applicants' 20-year experience in healing osteoporosis confirms that prior to proposing the claimed BAA none of the known compositions, separately or in combinations thereof, was able to solve the problem of organ calcification which takes place when preparations of the third category (promoters of bone mineralization) are used. Using drone brood separately, as well as using calcium compounds separately, is not effective in preventing and healing osteoporosis.

A new and unexpected technical result of the combined use of drone brood and calcium compounds in the proposed BAA is increased absorption of calcium by bone tissue and long retention thereof therein. This results in substantial lowering the risk of calcification of the body due to effective lowering the dosage of calcium compounds while maintaining same healing effect that was achieved earlier by the larger dosage of calcium. Combined use of drone brood and calcium compounds has not been known to date. Taking into account that recommendations for the use of a combination of drone brood and calcium compounds are not found in publications on drone brood and on calcium compounds and that this combination is inconsistent with modern views holding that calcium is not absorbed without vitamin D, such a combination of drone brood and calcium compounds in one product is believed to be non-obvious.

Thanks to the researches that led to the creation of the proposed BAA, there came a recognition that osteoporosis was a bone tissue cell death rather than the loss of calcium in a bone tissue cell. In such a case, there is no substance in the bone tissue to absorb calcium. This being so, the use of calcium preparations with a stimulator of calcium absorption, such as Vitamin D3, is believed to be dangerous for a patient because calcium forcedly absorbed by means of vitamin D3 in the intestine does not come to the bone tissue and is deposited in soft tissues, which results in calcification of vessels and consequently to strokes and cardiac infarctions, in kidneys, which results in the concrements formation, and so on.

It was uncovered in the course of those researches that the proposed BAA promotes the altogether growth of the bone matrix and facilitates retention of calcium in the body. The BAA triggers the process of the formation of a matrix ribonucleic acid (mRNA) required for protein synthesis in the bone tissue. As a result of that, a bone tissue cell fission starts. New bone tissue cells arise, and they intensively consume calcium to let the bone matrix grow, thus removing the problem of soft tissue calcification.

There is no calcium absorption stimulator in the proposed BAA since its use is believed to make no sense. The applicants believe that the digestive system is made sound, and if one or another component is not assimilated, that means that it is not supposed to be assimilated because there is no consumers for that component. The applicants hold that the digestive system works on tasks from other organs, and if there is no demand for one or another component, this component will not be assimilated by the digestive system. Consequently, the use of the calcium absorption stimulator contradicts the human physiology, that is why its use is impractical. What is more, it interferes with health because calcium forcedly absorbed by the digestive system ends up depositing in soft tissues which results in a hypercalcemia condition.

15-year examination of the city of Penza population conducted by the applicants uncovered high rate of hypermineralization—in about 7% of those examined. According to the results of field examinations conducted by the applicants using modern X-ray absorption osteometry, 8% of the population were diagnosed as having a hypercalcemia condition. With that in view, it was noted that various preparations, mainly calcium carbonate, were continuously administered to a substantial part of the population for no good reason. This factor is believed to be one of the major causes of hypermineralization. Numerous researches show that calcium preparation intake contributes to higher risk of calcium deposition in soft tissues and, as a result thereof, increases by 30% the risk of developing strokes and heart attacks. The applicants are unaware of the publications teaching how the calcification problem in the patients healed by calcium preparations can be solved. The problem cannot be solved by plain reducing the dosage of calcium, let alone that a physician cannot select the dosage at discretion because it is unattainable technically. The industry produces calcium preparations in certain discretization (for example, by 100 mg, 500 mg, 1000 mg, etc.), and for that reason it cannot make individualized dosage such as 339 mg for one patient and 511 mg for another.

For the time being, there is no approach that could define what calcium dosage is required for a specific person at this moment to rule out the risk of depositing calcium in soft tissues. On the other hand, the proposed BAA does not involve that risk. It was found that the combined use of drone brood and calcium compound in the proposed composition lowers side effect of calcium on the human body and widens the scope of persons whom the calcium preparations can be administered to.

The ratio of the calcium compound and drone brood according to the present proposal varies in a broad range and is defined by such individual parameters of a person as age, gender, health status, and other factors. The proposed BAA is an organic or inorganic calcium compound in amounts from 16.67 to 93.75 weight % and drone brood in the amounts from 6.25 to 83.33 weight %, wherein calcium is pharmacologically active and is presented by a compound from a series comprising calcium carbonate, calcium citrate, calcium gluconate, calcium aspartate, calcium amino acid chelate, calcium fumarate, calcium succinate, calcium ascorbate, calcium phosphate, or any combination thereof. The drone brood can be a homogenate in native, lyophilized, adsorbed or any other form made as a powder. The proposed BAA can be produced in the form of powder, tablets or capsules. The above-identified ranges make it possible to provide optimal intake of calcium and its most full assimilation in each individual with no risk of soft tissue calcification due to the claimed amount of drone brood in the BAA which secures a certain content of sulfhydryl groups of enzymes, as well as testosterone, progesterone, prolactin, and estradiol. Drone brood is rich in amino acids transporting calcium into a cell. Also needed for assimilating calcium are vitamins A, C, E, D and microelements of magnesium, copper, zinc, and phosphorus. They are presented in drone brood. Accordingly, all incoming calcium ends up completely assimilated by bone tissue. It turned out unexpectedly that holding calcium in the bone tissue for prolonged time is secured by a certain amount of sulfhydryl groups of enzymes in drone brood and by vitamins D and E slowing down the process of washing out the same. Besides, a long period of normal density of bone tissue can be explained by a substantially large amount of phosphorus in drone brood. It should be pointed out that the content of vitamin D in drone brood (about 850 IU per 100 g of drone brood) is too low to treat osteoporosis and other bone diseases.

It is noteworthy that it is just pharmacologically active compound of calcium that is used in the proposed composition. A pharmacologically inactive calcium compound does not fit. For example, calcium stearate cannot be used because this is a pharmacologically neutral substance which is of great utility as an excipient in manufacturing medicines (see, for example http://www.drugs.com/inactive/calcium-stearate-19.html), one of basic requirement thereto being its indifference in relation to the patient's body. Additionally, there is evidence (G. Gacs and D. Barltrop, Significance of Ca-soap formation for calcium absorption in the rat, Gut, 1977, 18, 64-68), indicating that fat acids, particularly stearic acid, prevent calcium from assimilating in the intestine. The cause of that—the formation of insoluble compositions (soaps), calcium stearate being one of those. Only 1% of calcium stearate is absorbed, the vast majority being washed out unchanged.

To produce the proposed food BAA, there is provided a powdered calcium compound selected from a series comprising calcium carbonate, calcium citrate, calcium gluconate, calcium aspartate, calcium amino acid chelate, calcium fumarate, calcium succinate, calcium ascorbate, calcium phosphate, or a combination thereof, in the amount of 16.67-93.75 weight %, it is mixed to homogeneity with drone brood, and the resulted homogeneous powder of light beige color and having humidity of 1-3.5% is subjected to tableting or capsulizing. There is Ig of the mixture in a tablet or a capsule. To prepare the BAA, drone brood is used in the form of lyophilisate. For producing lyophilisate, larvae of the drone brood are homogenized in a mixer. The homogenate is a thick yellow liquid of creamy consistency with a specific smell. The content of dry substance in the homogenate does not exceed 22.5%. The lyophilisate is produced by freezing the homogenate for 2-3 hours at minus 35-40° C. with subsequent sublimation in vacuum at residual pressure of 0.1-0.6 mm of mercury during 40-48 hours, bringing the temperature by the end of drying up to 25-30° C. The lyophilisate includes powder of beige to yellow color, of high hygroscopity and residual humidity of 1-3.5%. Thus, the way the lyophilisate is produced allows to preserve the component content of the dry substance of the drone brood homogenate unchanged except the moisture content.

### Examples of Producing the BAA

### Example 1

17 kg of powdered calcium gluconate is mixed with 83 kg of drone brood lyophilisate. The mixture is stirred to bring it to homogeneity, and the finished product is capsulized.

### Example 2

90 kg of powdered calcium gluconate is mixed with 10 kg of drone brood lyophilisate. The mixture is stirred to bring it to homogeneity, and the finished product is tableted.

For preventing osteoporosis, the regimen in taking the BAA is 1 capsule thrice a day for one month out of meal, the interval between courses being one month. During this time, calcium content in the serum does not lower and is maintained at a normal level. This result is confirmed by the following examples.

### Example 3

Patient, female, born in 1959.
Bone mineral density in the distal antibrachium was measured using densitometer DTX-200 of “Osteometr”, the index T being −1.4. Detected osteopenia. The patient was prescribed calcium gluconate by 1 g thrice a day. 6-month preventive course was administered. The examination after 6 months detected bone tissue mineral density (BMD) lowered down to T−1.6. After one month interval, calcium content in the serum was below normal. (For reference: a normal value of BMD varies from +1 to −1 of standard deviations from the peak bone mass, osteopenia of the 1^{st} degree has BMD from −1 to −1.5; osteopenia of the 2^{nd} degree range has BMD from −1.5 to −2; osteopenia of the 3^{rd} degree range has BMD from −2 to −2.5; osteoporosis of the 1^{st} degree is characterized by BMD of −2.5 and lower without fractures, while osteoporosis of the 2^{nd} degree has BMD of −2.5 and lower with bone osteoporotic fractures. The densitometer T-scale means a number of standard deviations above or below an average value of the bone mass peak. The T-criterion diminishes along with a gradual decrease of the bone mass with aging and is used for the BMD evaluation in adults.)

### Example 4

Same patient diagnosed with osteopenia and having BMD T of −1.6 was administered the BAA to food by 1 capsule thrice a day. A 6-month preventing course with the regimen of one-month-taking—one-month-gap was conducted. The BMD increased up to T−1.2, with the calcium content in the serum becoming normal. The examination repeated in three months detected no decrease of the calcium content in the serum.

An example of using a calcium preparation with vitamin D3, which demonstrates the risk of calcium deposition in soft tissues: Patient Mazen** R** Anat**, female, 59, diagnosed with systemic osteoporosis associated with rheumatoid arthritis, underwent surgery on two joints for coxarthrosis. Administered: a calcium preparation with vitamin D3, by 2 tablets twice a day in 3-month courses. The result of the treatment: a positive dynamics in one year with regard to BMD. Detected on the “after treatment” medical image is calcination in soft tissues.

Examples of using the proposed composition with no risk of having calcium deposited in soft tissues.

### Example 5

Nikol*** Svet*** Nik***, female, 45. Diagnosis: systemic osteoporosis. Administered: the proposed BAA, 2 tablets per day, nocte, in 3-month courses thrice a year. The result: the BMD increased, no soft tissue calcification observed.

### Example 6

Anis*** Lyub*** Nik***, female, 78. Diagnosis: osteoporosis. Administered: the proposed BAA, 2 tablets per day, nocte, in 3-month courses thrice a year. The result: improvement with regard to BMD with no soft tissue calcification observed.

### Example 7

Elistratova Lyubov'Ignatievna, female, 73. Diagnosis: systemic osteoporosis. Administered: the proposed BAA, 2 tablets mane and 3 tablets nocte, in 3-month courses with a month-long gap. The result: improvement with regard to BMD with no soft tissue calcification observed.

II. The Proposed Composition Surpasses its Counterparts in One More Important Technical Result: It Substantially Reduces the Risk of Fractures when Compared with the Use of Calcium Compounds with Vitamin D3.

When using the proposed BAA, no fractures were observed in 1000 persons for three years, i.e. 100% index of reducing the fracture risk has been achieved. Along with that, no evidence of soft tissue calcification was observed.

The combined use of drone brood and calcium in the BAA provides for more effective assimilation of calcium in the body because not only calcium comes into the body, but it is also kept in the bone tissue owing to drone brood. The feature of drone brood to enhance calcium assimilation in the body and to keep same in the bone tissue for a long time, i.e. to secure a prolonged effect after introducing calcium into the bone tissue has not been known from the prior art. Also, using calcium with drone brood allows reducing the dosage of the calcium compound which prevents the process of developing stones in kidneys from occurring and other calcium compound side effects, such as gastrointestinal upset.

In this way, achieved for the first time due to combined use of calcium compounds and drone brood in the claimed ratio was the object of not only saturating bones with calcium but prolonged keeping it therein as well. The risk of fractures reduced substantially, when compared with counterparts, since increasing the BMD by 5% lowers the fracture risk by 35%, additional antiresorptive properties of the BAA further lowering the fracture risk by 20%.

The applicants discovered that the proposed composition accelerates consolidation in fractures.

The problem of stimulating osteogenesis, along with finding the possibility of controlling the bone tissue regeneration is burning. This derives from a great number of patients of orthopedic and traumatologic profile with injuries resulting in suppressing reparative osteogenesis, as well as with locomotion system problems stemming from an imbalance of normal osteogenesis, such as osteoporosis.

It is well known that biological stimulation of reparative processes, including those in bone tissue, is performed by parenteral administration of protein preparations, anabolics, using pyrimidine- and purine-based compounds (pentoxyl, methyluracil, potassium orotate). With bone tissue in view, the biological stimulation is supplemented with preparations replenishing calcium deficit in the body and thus stimulating the phase of bone matrix calcification. They leave unaffected the speed of the formation of the bone protein matrix, which factor is the one defining the speed of osteogenesis as a whole.

In this regard, the closest analog of the proposed BAA is the Osteogenon preparation which is used for treating various forms of osteoporosis and promoting healing bone fractures (“Medication Registry—Encyclopedia of medications”, 2001, p. 656). For systemic osteoporosis, 2-4 tablets of Osteogenon are administered per os daily, one coated tablet containing 830 mg, and 1-2 tablets daily to promote healing bone fractures (Ibid.). The Osteogenon creators believe that the preparation, made of animal bone tissue, stops/diminishes bone tissue resorption (brakes osteoclasts) and stimulates osteogenesis (activates osteoblasts) (Ibid., p. 1110). However, the preparation is expensive, it is effective on long (from several months to a tear) use only, acerbation of kidney stone disease is possible, and the level of calcium and phosphorus control in urine is required.

It should be pointed out that the process of acting upon bone tissue in osteoporosis differs from that in fracture union. These are different processes of the bone tissue regeneration, and they take different courses. That is why it would be wrong to conclude that a certain composition is useful and effective for promoting the fracture union on the ground that that composition is used for healing osteoporosis. For example, administering bisphosphonates, which are widely used with regard to osteoporosis, for healing fractures may result in failure to heal the fracture. Same relates to calcitonin salmon (brand name Miacalcic). Miacalcic is used actively in healing osteoporosis, but it is totally inappropriate for healing fractures. What is more, it is harmful for fractures because it slows down substantially the speed of the union. There can be more examples of this type of mismatch.

The technical result to be achieved by the present invention in this aspect thereof is promoting the fracture union and osteosynthesis, and, thus, reducing the time of rehabilitation and time of disability.

This result is also attained by administering drone brood and calcium compound. According to the invention, the composition for healing fractures comprises drone brood and calcium compound in the above-defined ratio, the composition being made in a tablet or capsule form, the calcium compound including one of the above-identified group of preparations or any combination thereof.

On a nationwide scale, work losses caused by bone injuries (even when estimated very roughly) amount to a huge number of scores of millions workdays. In Penza only, for example, about 50,000 people apply to hospitals annually with various kinds of traumas. About 60% of them have traumas that do not require hospitalization (these are not displaced, or insignificantly displaced, fractures) and about 40% have traumas requiring hospitalization, including 10% that require surgery. And if it were possible to accelerate fracture unions even by a third, it would enormously benefit people and society.

Currently, in connection with delayed union, a trend is noticed for increasing the time of immobilization of fractures, which results in larger time of rehabilitation and disability.

The fracture union is known to take place with a number of complex topical and general biological transformations. Five phases of bone tissue regeneration can be defined as follows.

At the First phase, immediately after the injury, an embryonic (mesenchymal) tissue begins to form, and a kind of jelly “primary gum” is formed from hematoma (edematous fluid and fibrin) in the area of bone fracture.
At the Second phase, the differentiation of hematoma cellular elements and the formation of fibrous structures occur to produce cellular-fibrous tissues serving a base which the bone stock is then deposited on.
At the Third phase, the deposition of bone tissue takes place. In collagenous fibers of the connective tissue callus, sites of induration start emerging yielding continuous mass due to protein deposition. Formed based on that are primitive bone-like trabeculae, first—sporadic and then—as a dense network.
At the Fourth phase, formation and calcification of the bone callus takes place. The callus calcification progresses primarily at the expense of calcium in blood where it comes from the whole skeletal system, including areas immediately adjacent to the bone fracture area.
At the Fifth phase, callus restructuring takes place where less mature bone structures are replaced with trabecular bone tissue, and adaptation to load conditions comes about. The bone callus is being restructured according to functional requirements, and resorption of some structures along with creation of, and strengthening, the others takes place. The restructuring of the final callus can last months and even years, depending on the placement of consolidated bone fragments, the size of the callus and the correspondence of the axis of the extremity to bone load functional requirements.

Various publications point out that the bone fracture recovery can be interrupted at any of the stages of forming the bone callus—by reason of large hematoma, poor fragment placement, osteoporosis, calcium deficit in the body (which can result from improper life habits, such as smoking, alcohol, inactive lifestyle, sun exposure deficit), wrong diet (inadequate amount of calcium- and phosphorus-containing food). All these factors affect the consolidation processes and bone density. With that in view, researches aimed at learning the mechanisms of normalization or boosting the bone consolidation processes, as well as developing respective remedies, are of great scientific and practical interest.

It was uncovered in the course of researching the proposed composition that it accelerates fracture consolidation due to supporting activity of osteoblasts engaged in building bone tissue.

This idea was tested in practice. Below are the illustrations of the research:

1. Moderately Severe Fractures.

Selected for the research were 300 patients (112 males and 188 females) at the age of 18-62 with most commonly encountered fractures—a radial bone distal meta-epiphysis fracture and an ankle joint lateral malleolus fracture.
The patients were divided into three groups:
the 1^{st} group (122 patients) was on calcium and drone brood (designated below as BAA), 3 tablets twice a day;
the 2^{nd} group (103 patients) took “Calcium D3 Nikomed”, one tablet twice a day; and
the 3^{rd} group (75 patients) was administered no calcium preparations.
All patients underwent X-ray analysis prior to the plaster application and in three weeks after taking same off. All patients were given food rich in calcium. Average fracture immobilization time of 4 weeks was selected.

### Roentgenologically:

In all patients of the 1^{st} group taking BAA, bone callus was noted in 4 weeks.
In patients of the 2^{nd} group taking “CaD3 Nikomed”, subconsolidation is noted, no bone callus observed, it becoming noted only on the 4^{th}-5^{th} week of immobilization.
In the 3^{rd} group, where patients took no calcium preparations, no bone calluses noted, they can be observed on the 5^{th} week only.

Those results show that the patients taking BAA can start rehabilitation process in 3 weeks after the trauma and resume working in a month after the trauma.

The patients taking “CaD3 Nikomed” could start rehabilitation only in 4-5 weeks after the trauma and resume working in 1.5 months.
The patients taking no calcium preparations started rehabilitation only in 1.5 months after the trauma and resumed working in 2 months after the trauma.

2. Severe Fractures.

Selected for the research were 160 patients (70 males and 90 females) at the 18-62 age range suffering from severe fractures—spinal fracture and pelvic bone trauma. Average time of fracture immobilization—12 weeks—was selected.
The patients were divided into three groups:
the 1^{st} group (90 patients) was on calcium and drone brood (designated below as BAA), 3 tablets twice a day;
the 2^{nd} group (50 patients) took “Calcium D3 Nikomed”, one tablet twice a day; and
the 3^{rd} group (75 patients) was administered no calcium preparations.
All patients underwent X-ray analysis immediately after the fracture and in 8-10 weeks after the fracture. All patients were given food rich in calcium.

### Roentgenologically:

In all patients of the 1^{st} group taking BAA, bone callus was noted in 8-9 weeks, i.e. it occurs 2-4 weeks prior to the average time of consolidation for this type of fractures.
In patients of the 2^{nd} group taking “Ca D3 Nikomed”, subconsolidation is noted, no bone callus observed, it becoming noted only on the 11^{th}-12^{th} week of immobilization.
In the 3^{rd} group, where patients took no calcium preparations, bone calluses can be observed on the 12^{th} week only.

Those results show that the patients taking BAA can start rehabilitation process and resume working earlier than the patients from two other groups.

The patients taking “Ca D3 Nikomed” could start rehabilitation only in 11 weeks after sustaining a severe trauma.
The patients taking no calcium preparations started rehabilitation only in more than three months after sustaining a severe trauma.

Examples of the Composition Profile:

1) Calcium citrate 200 mg+drone brood 100 mg;
2) Calcium gluconate 300 mg+drone brood 200 mg;
3) Calcium carbonate 100 mg+drone brood 50 mg;
4) Calcium carbonate 50 mg+calcium lactate 50 mg+drone brood 150 mg;
5) Calcium ascorbate 300 mg+drone brood 300 mg;
6) Calcium phosphate 1000 mg+drone brood 500 mg;
7) Calcium fumarate 1 mg+drone brood 1 mg;
8) Calcium carbonate 10 mg+drone brood 2 mg;
9) Calcium citrate 1000 mg+drone brood 200 mg;
10) Calcium citrate 15 mg+drone brood 5 mg;
11) Calcium gluconate 40 mg+drone brood 20 mg;
12) Calcium carbonate 10 mg+drone brood 3 mg;
13) Calcium fumarate 500 mg+drone brood 150 mg;
14) Calcium citrate 100 mg+drone brood 1000 mg;

As it follows from the above examples, the claimed preparation can be offered in various ratios of the components thereof—drone brood and calcium compound, the extent of the technical effect achieved depending on a particular individual and its size, as well as on the gradations of the malady.

Based on the above, a conclusion can be come to that the use of the proposed composition in patients with fractures is favorable to the substantial reduction of the time of immobilization. This makes it possible to perform rehabilitation earlier which results in coming back to work earlier which undoubtedly has an important socioeconomic impact.

Summing up, it is believed that the use of a combination of drone brood and calcium compound for promoting fracture consolidation was not known prior to the current proposal.

III. It was Also Uncovered that the Use of the Claimed Composition Facilitates Filling Cavities in Bone Tissues.

For a long period of time, examining more than 10,000 patients in the Penza region, physicians were noting areas of bone loss. A term ‘cavitary lesion’ was suggested to be used for naming such areas. These pathological cavitary lesions have nothing to do with the cavities serving a case for the spinal cord and are observed in people of various age, including even children.

### Clinical Examples

Patient L., male, 12.
Complaints: frequent fractures (1-2 times annually during three years).
Findings: height 158 cm, weight 60 kg. Regular physique. Excessive subcutaneous fat. Visceral organs unremarkable. The child was repeatedly examined by specialists. Cavitary lesions in trabecular segments of bones are noted on X-ray osteodensitometry.
Patient Nikol*** Svet*** Nikol***, female, 45:
Roentgenologically: a cavitary lesion is noted in the elbow bone.

Until recently, medicine did not know how to deal with such areas. It was noted when examining patients that those areas have a tendency to increase, even though the patients were taking various preparations to strengthen bone tissue: bisphosphonates, strontium ranelate, calcitonin, preparations of calcium and vitamin D3, and others. It was clear that those areas are a great threat to patients because their development resulted in further fractures, the place of fracture coinciding with the area.

It is significant that the cavitary lesions in question are not believed to be directly associated with osteoporosis because they are found in both people suffering from osteoporosis and people free of osteoporosis. On the other hand, there are people with osteoporosis in whom these cavitary lesions are not found. Reviewing publications and participating in various symposiums and conferences failed to find a mechanism of acting upon those cavitary lesions (focuces). It is believed that these cavitary lesions are not directly associated with osteoporosis and are of a different nature. In practice, applicants dealt with patients who were not diagnosed with osteoporosis based on densitometry data and WHO recommendations because their BMD was more than −2.5 SD, up to −2 SD. Those patients enjoyed normal density of the bone tissue and, nevertheless, had cavitary lesions in the bone tissue.

Set forth below are the results of a number of osteometric observations of the patients who, based on BMD evaluation, was not diagnosed earlier with osteoporosis—see examples 8 and 9.

### Example No. 8

Patient A., female. The BMD lowered on the T-score from −2.1 to −2.3 SD which fits the range of osteopenia of the 3^{rd} degree according to the WHO classification. However, found in this patient's trabecular segment of the elbow bone was cavity of 2×4 mm.

### Example No. 9

Patient b., female. The level of BMD corresponds to osteopenia of the 2^{nd} degree (−1.8 SD). However, trabecular segments of bones comprise areas with pronounced demineralization, the ones called cavities.

An increase of number and size of cavitary lesions in metaphyseal areas of bones is not a specific characterization of calcium deficit Set forth below are examples of the patients who were diagnosed with cavitary lesions (cysts) but did not suffer from calcium deficit and, to the contrary, hypermineralization, i.e. calcium deposition in soft tissues because of overuse thereof was diagnosed in them.

### Example 10

Patient FAD, female, 64.
Administered a year-long course of “Calcium D3 forte” made by Nicomed.
Noted: hypermineralization—calcination of soft tissues.
Roentgenologically, calcification of soft tissues is clearly noted which attests to hypermineralization. Nonetheless, the patient has cavitary lesions.

### Example 11

Patient Z., female, 66.
Administered a 9 month-long course of “Calcium D3 forte” made by Nicomed.
Diagnosed with hypermineralization—calcination of soft tissues.
Nonetheless, the patient has cavitary lesions.

### Example 12

Patient ZI, female, 70.
Administered a 12 month-long course of “Calcium D3 forte” made by Nicomed.
Diagnosed with hypermineralization—calcination of soft tissues.
Nonetheless, the patient has large cavitary lesions.

The applicants are believed to be the first to create a composition intended for acting upon the cavitary lesions in question. The use of the claimed composition allows the human body to fill the cavities where necessary. It is also worth noting that owing to drone brood in the composition the required calcium is assimilated faster and does not leave side effects, such as calcination of blood vessels or kidney.

When researching the proposed composition, various methods of administering the same were used: rectal suppositories, sublingual and swallowed tablets, and intramuscular and intravenous injections.

A total of more than 10,000 people were examined who took calcium and drone brood in various forms. The analysis performed made it possible to come to the following conclusions: 1. The result achieved does not depend on the form and way the composition entered the body. 2. The result achieved does depend on the amount of calcium and drone brood entering the body, on the duration of taking the both components, and on the age and individual peculiarities of the body.

Calcium in the body was checked using a DTX-100 densitometer of Osteometer MediTech, Inc. by measuring distal forearm bone density (BMD), normal findings being from +1 to −1 of standard deviation from a peak bone mass in the T-criterion indication. As in the above, osteopenia of the 1^{st} degree has BMD from −1 to −1.5; osteopenia of the 2^{nd} degree range has BMD from −1.5 to −2; osteopenia of the 3^{rd} degree range has BMD from −2 to −2.5; osteoporosis of the 1^{st} degree is characterized by BMD of −2.5 and lower without fractures, while osteoporosis of the 2^{nd} degree has BMD of −2.5 and lower with bone osteoporotic fractures. The densitometer T-scale means a number of standard deviations above or below an average value of the bone mass peak. The T-criterion diminishes along with a gradual decrease of the bone mass with aging and is used for the BMD evaluation in adults. Measuring BMD can be visualized—the lower the mineral density, the darker the area. Black color designates cavity, and as the mineral density increases the color changes from blue to red to yellow to white.

Also used was the calcium Sulkowitch test making it possible to approximately evaluate the level of calciuria and, consequently, of hypercalcemia. The test is performed as follows: 2 ml of a reagent consisting of 2.5 g of oxalic acid, 2.5 g of ammonium oxalate, 5 ml of glacial acetic acid and distilled water to make 150 ml, is added to 2 ml of 24-hour urine. A light milky precipitate of calcium oxalate indicating a normal level of calcium in blood is formed in the test-tube in 0.5-1 minute after the mixing. If so, the reaction is considered weakly positive (+). In the case that the level of calcium in blood is increased, a clear and massive cloudiness appears immediately (++). When the level of calcium is above 13 mg %, an extreme cloudiness appears (+++). In this way, a safe amount of calcium entering the body was secured, or—in the case of a dangerous deficit thereof—the admission of calcium to the body resumed.

The monitoring of the patients whom drone brood was administered was performed by examining the patients and blood and urine tests. In the course of monitoring, taking high dose of drone brood, up to the point of complete moving to a drone brood diet, showed no adverse affect.

Examples of acting upon cavitary lesions with the proposed composition:

### Example 13

Examined from 2007 through May of 2013, at the clinic houses of Penza regional clinical children's hospital, were 37 children and teenagers (25 boys and 12 girls) with low mineral bone density and fractures of long bones of distal segments of extremities who presented the primary population. Fractures of upper extremities prevailed (28 cases against 9 cases for lower extremities). Included in the research were only children and teenagers at the ages of 10-18 having BMD lower than −2.0 SD, bone fractures and cavities in trabecular sections of bones. Low bone mineral density was diagnosed based on the lower than −2 indications on the Z-scale in terms of BMC, BMD, the presence of the main criterion—fracture(-s), and statural-weight values.

Excluded from the monitoring were children with various general diseases, brittle bone disease, tubulopathy, hyperparathyroidism, as well as those taking glucocorticoids. The examining of the patients comprised collecting medical history, examination, and general clinical, laboratory and biochemical studies. Estimated with the use of roentgen methods were bone markers: general and ionized calcium, inorganic phosphorus, alkaline phosphatase, calcidiol 25(OH)D in blood serum, parathormone, osteocalcin, hormones TSH, T3 and T4. Vitamin D deficit was established if there were less than 20 ng/ml of 25(OH)D in blood. Testing bone tissue mineral density (BMD) was conducted by the method of X-ray absorption using a DTX-100 densitometer of Osteometer MediTech, Inc., prior to and 9-10 months after the treatment. Three groups of the causes of repeated fractures were identified in the course of the in-depth monitoring: deficit of vitamin D (1^{st} group, 11 children), cavitary lesions in trabecular segments (2^{nd} group, 7 children), and low bone mineral density (3^{rd} group, the remaining 19 children).

Two examples of treating children with cavitary lesions in the forearm trabecular segments.

### Example 14

Patient P., a boy, 15, had cavitary lesion in the distal segment of the elbow bone. After 2 year use of the claimed composition, the lesion was filled.

### Example 15

Patient AA., a boy, 12. Roentgenologically, cavitary lesions are found (as black dots against the blue background of the bone tissue) in the spoke and elbow bones. After ten months of using the claimed composition, the lesions were filled.

### Example 16

Patient AAA., female, 54, with BMD of −2.0 SD. Before approaching the applicants, the patient was taking Calcium D3 of Nicomed comprising 1250 mg of calcium carbonate and 200 IU of vitamin D3, 1 tablet twice daily for 12 months. Still, cavitary lesions were visible on the film. Then, the claimed composition of calcium carbonate 500 mg along with 500 mg of drone homogenate daily orally was administered. After nine months of taking the composition, the cavitary lesions were filled, the BMD being −1.2SD.

### Example 17

Patient BBB., female, 55, with BMD of −1.9 SD. Before the exam, the patient was taking Calcium D3 of Nicomed (1250 mg of calcium carbonate with 200 IU of vitamin D3), 1 tablet twice daily for 12 months. After the treatment, a film shows cavitary lesions. After nine months of taking parenterally the claimed BAA (500 mg of calcium gluconate and 500 mg of drone homogenate daily) the cavitary lesions were filled, the BMD being −1.5SD.

Patients AAA. and BBB. have approximately equal cavitary lesions.

It was established that, when drone brood is administered, important is not so much the amount of drone brood as the rhythmicity of taking same, and with that in view, the more frequently the patient takes drone brood, the lower dosage can be used, and, respectively, the less frequently drone brood is taken, the higher one-time doses are required.

Example 18 illustrates duration of intake of the preparations of calcium and drone brood homogenate.

Patient RLG, female, was taking tablets Calcium D3 of Nicomed (one tablet comprising 1250 mg of calcium carbonate and 200 IU of vitamin D3, twice a day) for more than one year. Yet, a cavitary lesion is still detectable on a film. After nine months of taking daily 1 g of calcium citrate and 500 mg of drone brood, the cavitary lesion was no more detected. Then, during two months, the patient was administered 500 mg of drone brood daily. BMD remained unchanged, cavitary lesions are absent. After that, during three months the patient took 1 g of calcium citrate and 500 mg of drone brood daily which resulted in the increase of BMD, with cavitary lesions undetected. Then, during three months the patient was administered 0.5 g of calcium citrate and 1000 mg of drone brood daily. BMD was unchanged, with cavitary lesions undetected. After that, the intake of calcium preparation and drone brood was cancelled for three months. Cavitary lesions were detected. Conclusion: calcium preparations and drone brood should be administered for long time with intervals of no more than a month.

### Example 19

Female patients, 51-70, with compromised bones were administered same dose of drone brood—500 mg daily. Also administered was calcium gluconate, the regimen therefor being two 500 mg tablets orally, one hour before meal thrice a day. The preparations were taken in three-month courses, each followed by a one-month interval. Before and after the three-month courses up until the end of the treatment, BMD and the dynamics of cavitary lesion dimensions were evaluated for all the patients by X-ray absorption method using DTX-100 densitometer. The results of the evaluation after 12-month treatment are presented in Table 1.

TABLE 1
Bone tissue treatment comparative efficiency after 12 months

| Number | BMD | | | Cavitary lesion dynamics | | |
|---|---|---|---|---|---|---|
| of | Better, | Unchanged, | Worse, | Positive, | No change, | Negative, |
| evalu- | abs. | abs. | abs. | abs. | abs. | abs. |
| ations | numb. | numb. | numb. | numb. | Numb. | numb. |
| 25 | 21 | 4 | 0 | 25 | 0 | 0 |

The effect of the decrease of cavitary lesions was due to contemporaneous intake of calcium and drone brood.

Subject to individual peculiarities of the body, the amount of calcium consumed with food, and the number and size of cavitary lesions, the desired result can be reached sooner or later, but it is reached anyway. In practice, substantially significant results were achieved for 100% of all the patients at reasonably long-term ingestion.

When conducting the trial of the proposed composition, the preparation was used in tablets and intramuscular or intravenous injections.

IV. Applying the Claimed Composition to Treating Arthritis and Arthrosis.

The additional technical result which the claimed composition pursues to reach is the acceleration of treating arthritis and arthrosis and the increase of effectiveness, while eliminating at the same time the chances of advent of hypercalcemia.

Arthrosis and arthritis (international term—osteoarthritis) is a widely present group of diseases having various causes and characterized in the development of dystrophic and inflammatory transformations in bone tissue. Currently, most widespread programs for dealing with “polyarthritis” use steroid and non-steroid anti-inflammatory and analgesic preparations. They are ineffective, providing symptomatic effect only, and substantially unhealthy. Also, those preparations do not take age-related causes of advent of arthritis and arthrosis into account.

The applicants found out that one of the causes of arthritis and/or arthrosis is the development of cavitary lesions in the bone tissue. The cause of the development can vary. In the course of forming cavitary lesions, normal metabolism of the bone segment located next to the cavitary lesion declines. As a result, as an osteoclast destroys a bone cell, the mineral matrix has no way to go because the cavity lesion prevents the matrix from being excreted from the bone tissue, the matrix remains in the bone, and arthrosis emerges. In other words, the cause of arthrosis is not the result of changes in the muscular frame and a method of stabilizing the body, but rather preventing, by the cavitary lesion, the destroyed mineral matrix from being excreted from the bone.

The same cause is in developing arthritis—an inflammation in the bone tissue. A cavitary lesion prevents bone cell waste products, as well as dead cells, from being excreted from the bone tissue. As a result, an inflammation—arthritis—emerges.

As the applicants found out, the use of the claimed composition makes it possible to bring the process of developing arthrosis and arthritis to stop and to make the process reverse. In as little as one month of taking drone brood with one of the above-mentioned calcium compound and following a diet with a large content of calcium, aching pain in joints goes away which allows patients to cancel the use of non-steroid anti-inflammatory medications. Continued intake of the claimed composition results in even further filling the cavitary lesions to thus make it possible to improve metabolism of the bone tissue behind the cavity. The process of arthrosis development lets up, joint movability improves, pain when moving decreases. The experience of using the claimed composition by more than a thousand patient with arthritis and arthrosis shows that a substantial progress in them arrives after 6-9 months of treatment. The patients unable to move because of agonizing pain started moving around and taking care of themselves. Used in the course of the experiments were tablet, capsule or powder forms of the claimed composition.

When using the preparation comprising calcium compounds and drone brood, the dosage was two tablets thrice a day to secure positive dynamics of the main clinical scores, instrumental and chemistry values. Frequency of the pain syndrome and its severity was three times smaller after 1-2 months. Also noted were positive dynamics in the form of pain disappearance or decrease, mineral density increase, and filling of cavities or decrease of their size.

Set forth below is a comparative characteristic of a combination treatment of polyarthritis with secondary systemic osteoporosis using the claimed composition and Calcium-D3.

The object of the trial was a comparative evaluation of the results of 3-month treatment of polyarthritis with secondary systemic osteoporosis using the claimed composition and Calcium-D3.

Matter and Method of Trial:

Patients' criteria for the inclusion into the trial were: no gender limitation, between 45 and 65, painful ambulation (at more than 40 mm according to Visual Analog Scale (VAS)), systemic osteoporosis associated with polyarthritis (grade II-III per Kellgren-Lawrence grade (K-L system)). Patients' criteria for the non-inclusion into the trial were: painful ambulation at less than 40 mm according to VAS, rheumatoid polyarthritis of grade IV per K-L system, surgical management of rheumatoid polyarthritis less than six months prior to the trial, individual intolerance to the preparations under the trial, exacerbation of chronic somatic diseases.

The first group comprised 20 patients administered with the claimed composition—two tablets (each containing 100 mg of the adsorbed drone brood homogenate and 200 mg of calcium citrate) thrice a day. The second group comprised 20 patients for whom Calcium D3 in tablets was administered, one tablet in the morning and one in the evening, the tablet comprising 1250 mg of calcium carbonate and 200 IU of vitamin D3, the patients and their parents having no bone fractures in their medical history.

Serving as criteria of the efficacy of the preparations were: the impact on the activity of the inflammation process, the effect on the pain syndrome per VAS according to the evaluation of both the patient and physician, the number of painful joints (PJN), the number of swollen joints (SJN), joint range of motion at the admission and at the discharge, comparative estimate of the sedimentation rate (ESR) and C-reactive protein (CRP) at the admission and at the discharge.

The trial was conducted in the patients who started treatment for the secondary systemic osteoporosis associated with polyarthritis at the department of rheumatology and then continued same outpatiently during 2.5 months. The patients were subjected to medical examination, evaluation of pain per VAS, bone densitometry, radiological examination of hands and feet, and laboratory assessment of acute-phase blood protein, rheumatoid factor, calcium in blood, and alkaline phosphatase, the trial using state-of-the-art equipment and adequate standard procedure.

According to the bone densitometry, all the patients demonstrated lower BMD by T-criterion down to −2.4 SD as to the spinal column, down to −2.1 SD with regard to the right femoral neck, and down to −⁺2.6 SD as far as the left femoral neck is concerned which showed signs of systemic osteoporosis. Findings of the trial were entered into patients' individual charts and subjected to statistical analysis.

Trial Results.

Over the span of the trial, the amount-of-pain index per VAS on walking for the patients of the 1^{st} group in the course of the treatment with the claimed composition showed by the 3^{rd} month the decreasing tendency from 72 mm to 53 mm, i.e. by 26%, and when evaluated by a physician—from 63 mm to 43 mm, i.e. by 32%. In the course of the treatment with Calcium—D3, the same index for the 2^{rd} group had the decreasing tendency from 76 mm to 73 mm, i.e. by 3.9%, and when evaluated by a physician—from 62 mm to 60 mm, i.e. by 3.2%. The amount-of-pain index on palpation of the affected joint in the course of the treatment with the claimed composition decreased in two weeks by 27% as compared with 5% for Calcium-D3. The joint range of motion on knee and hip bending and unbending remained unchanged for both groups. The PJN decreased in three months from 7.0 down to 2.5, i.e. by 64%, with the use of the claimed composition, as compared with 6.4 down to 5.9, i.e. by 8%, when using Calcium-D3. The SJN caused by the secondary synovitis associated with rheumatoid arthritis decreased in three months from 3.7 down to 2.8, i.e. by 24%, for the claimed composition and from 3.8 down to 3.5, i.e. by 8%, for Calcium-D3. ESR for the 1^{st} group patients (taking the claimed composition) dropped from 44 mm to 28 mm in three months, i.e. by 33%, and CRP from 46 to 23 in three months, i.e. by 50%. In the 2^{nd} group (using Calcium-D3), ESR decreased from 38 mm to 35 mm in three months, i.e. by 7.9%, and CRP from 42 to 40 in three months, i.e. by 4.8%. Numbers for alkaline phosphatase and calcium in blood remained unchanged over the course of three months.

Accordingly, the analysis of the primary factor dynamics, which is the criteria of the efficacy of the treatment with the use of the claimed composition, showed that those factors improved over the course of the trial on the average of 30.7% as compared with the use of Calcium-D3. This makes it possible to appraise the treatment using the claimed composition as an effective one.

With regard to side effects in the course of the treatment using the above-mentioned compositions, pain in the epigastric region and bowel disorders were noted in two patients in the 2^{nd} group on top of taking Calcium-D3, as well as the development of hypercalcemia in the same group.

### CONCLUSION

the claimed composition when used in patients having polyarthritis with secondary systemic osteoporosis is tolerated well, results in substantial reducing pain on movement and palpation, and increases motion activity of the patients. Clinically significant positive effect of the composition is noted in three months of use. The intensity of the positive healing effect of using the claimed composition in patients with polyarthritis with secondary systemic osteoporosis developed as a result of arthritis maltreatment exceeds substantially the same when Calcium-D3 is used.

Also, it was uncovered that the claimed composition is helpful in treating paradontosis. It was noted that osteoporosis starts in the lower portion of a jaw, persistent parodontitis (inflammation of the gum tissue) being a marker of the development of osteoporosis. The lower portion of a jaw is the most loose bone tissue in the human body. Bone tissue loss occurs in the jaw bone area adjacent to the base of a tooth. Because of that, the tooth taking a bite of rough food becomes wobbly and tears the gum. It starts bleeding. Using astringent remedy results in the tightening of the gum surface (the gum diminishes in size), and parodontitis goes away for a while. Then, new food loosens the tooth again, and it tears the gum anew. This proceeds until the tooth is lost. The use of the claimed composition makes it possible to strengthen the base of a tooth and thus prevent the gum from bleeding and paradontosis from developing.

Summing up, it is appropriate to point out that the components of the claimed BAA in the form they are used therein cannot be found in nature. This relates to the drone brood, that can be used in this invention only after it is processed in the above-described way, and to some of the calcium compounds used, such as calcium citrate, calcium gluconate, calcium aspartate, calcium fumarate, etc. It is also worthwhile to emphasize that though there exist prior art publications disclosing the use of drone brood and calcium—separately—to deal with compromised bone, no prior art was found suggesting their combined use. What is more, the inventors of this invention uncovered that the combined use of drone brood and calcium results in a synergistic effect, which is extrinsic to (uncharacteristic for) each of the components. Specifically, it leads to lesser dosage of calcium required, better assimilation of calcium by the body, and, last but not least, substantially reduces the risk of calcium deposition in soft tissues (untargeted areas)—the side effect of using calcium administered by physicians which makes patients stop taking calcium. With these arguments in view, the proposed invention is believed to be patently eligible (patentable) and nonobvious.

1. A biologically active additive composition for preventing and healing bone diseases, the composition comprising:
a pharmacologically active calcium compound chosen from a series comprising calcium carbonate, calcium citrate, calcium gluconate, calcium aspartate, calcium amino acid chelate, calcium fumarate, calcium succinate, calcium ascorbate, calcium phosphate, or any combination thereof, in the amount of 16.67-93.75 weight %, and
drone brood in the amount of 6.25-83.33 weight %,
to thereby make it possible to substantially reduce risk of calcium deposition in untargeted organs when dealing with osteoporosis, arthritis, arthrosis, paradontosis, as well as to accelerate the union of fractured bones and to fill bone cavities resulted from the bone diseases.

2. The composition as claimed in claim 1, wherein the drone brood includes adsorbed homogenate thereof.

3. The composition as claimed in claim 1, wherein the drone brood includes lyophilisate thereof.

4. A method of preparing the composition claimed in claim 1, comprising the steps of:
providing drone brood larvae and the calcium compound;
performing lyophilisation of drone brood by means of:
homogenizing the drone brood larvae in, for example, a mixer;
freezing the homogenate for 2-3 hours at −35÷−40° C.;
subjecting the homogenate to vacuum sublimation at a residual pressure of 0.1÷0.6 mm Hg during 40-48 hours;
gradually raising temperature in the course of the sublimation up to 25÷30° C. by the end of the sublimation;
milling the calcium compound; and
mixing the milled calcium compound with the drone brood lyophilisate to homogeneity.

5. The method as claimed in claim 4, wherein powder, tablets or capsules are made of the mixture of the milled calcium compound and drone brood lyophilisate.

6. A method of preparing the composition claimed in claim 1, comprising the steps of providing an adsorbed homogenate of drone brood; providing the calcium compound; milling the calcium compound; and mixing the milled calcium compound with the adsorbed homogenate of drone brood to homogeneity, whereby producing powder, tablets or capsules therefrom is become possible.

7. The method as claimed in claim 6, wherein the adsorbed homogenate of drone brood is prepared by:
extracting drone brood from a honeycomb and milling or grinding same;
providing one part of drone brood homogenate and 3-30 parts of an adsorbent by mass, wherein said adsorbent is selected from the group comprising lactose, glucose, fructose, or any combination thereof;
mixing the one part of drone brood homogenate with the 3-30 parts of adsorbent;
vacuum drying the mixture of the homogenate and adsorbent in a vacuum installation,
the vacuum drying starting no later than 55 minutes after extracting drone brood from the honeycomb,
progressing drying without heating the mixture and keeping pressure in the vacuum installation no less than 1 mm Hg, and
ending drying at the humidity of the product not exceeding 1.5%.
